(19) United States
(12) Patent Application Publication
(10) Pub. No.: US 2003/0144559 A1
(43) Pub. Date: Jul. 31, 2003

(54) Process for the preparation of aldehydes from olefins by hydroformylation

(75) Inventors: Dieter Hess, Marl (DE); Dirk Roettger, Recklinghausen (DE); Detlef Selent, Berlin (DE); Armin Boerner, Rostock (DE)

Correspondence Address: OBLON, SPIVAK, MCCLELLAND, MAIER & NEUSTADT, P.C., 1940 DUKE STREET, ALEXANDRIA, VA, 22314, US

(73) Assignee: Degussa AG, Marl (DE)

(21) Appl. No.: 10/292,448

(22) Filed: Nov. 13, 2002

Related U.S. Application Data
(63) Continuation of application No. 09/708,646, filed on Nov. 9, 2000, now abandoned.

(30) Foreign Application Priority Data
Nov. 12, 1999 (DE) 199 54 721.1

(51) Int. Cl. C07C 45/50; C07C 29/15

(52) U.S. Cl. 568/451; 568/909

(57) ABSTRACT

A process for the catalytic hydroformylation of olefins having from 3 to 24 carbon atoms, where the catalyst used comprises a metal of transition group 8 of the Periodic Table in the presence of a ligand represented by formula I:

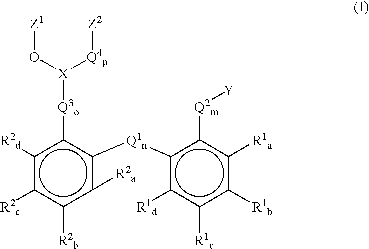

wherein

X=As, Sb, P;

R¹_{a-d}, R²_{a-d}=H, aliphatic or aromatic hydrocarbon radical, aliphatic or aromatic alkoxy group, in each case having from 1 to 25 carbon atoms, where R¹_{a-d} and R²_{a-d} can each be identical or different;

Q¹, Q², Q³, Q⁴=O, S, NR⁷, CR⁷R⁸, where R⁷ and R⁸ can be identical or different and can have one of the meanings of R¹ₐ, n, m, o, p=0 or 1, with the proviso that either o or p is 1;

Y=—O—R₅, —COOR₅, —COOM, —SR⁵, —NR⁵R⁶, —N═CR⁵R⁶—COR⁵, —CONR⁵R⁶, —F, —Cl—Br, —I, where R⁵ and R⁶ can be identical or different and are H, an aliphatic or aromatic hydrocarbon radical having from 1 to 25 carbon atoms and M=H, Li, Na, K or NH₄; and

Z¹, Z²=substituted or unsubstituted aliphatic or aromatic hydrocarbon radical having from 1 to 75 carbon atoms, where Z¹ and Z² may be covalently linked.

### BACKGROUND OF THE INVENTION

1. Field of the Invention

The present invention relates to a process for preparing aldehydes by hydroformylation of olefins or olefin mixtures in the presence of a catalyst comprising a metal of transition group VIII and a functionalized phosphonite ligand.

Aldehydes can be prepared by catalytic hydroformylation of olefins having one less carbon atom (oxo process). The hydrogenation of these aldehydes gives alcohols which are used, for example, for preparing plasticizers or as detergents. Oxidation of the aldehydes gives carboxylic acids which can be used, for example, for preparing drying accelerators for surface coatings or as stabilizers for PVC.

2. Background of the Invention

The type of catalyst system and the optimum reaction conditions for the hydroformylation depends on the reactivity of the olefin used. A concise overview of hydroformylation, examples of catalysts and their fields of application, current industrial processes, etc., may be found in B. Cornils, W. A. Herrmann (Ed.), “Applied Homogeneous Catalysis with Organometallic Compounds”, VCH, Weinheim, New-York, Basel, Cambridge, Tokyo, 1996, Vol. 1, pp. 29-104. The dependence of the reactivity of the olefins on their structure is described, for example, by J. Falbe, “New Syntheses with Carbon Monoxide”, Springer-Verlag, Berlin, Heidelberg, New York, 1980, p. 95 ff. The differing reactivity of isomeric octenes is also known (B. L. Haymore, A. van Hasselt, R. Beck, Annals of the New York Acad. Sci., 415 (1983), pp. 159-175).

The various processes and catalysts make it possible to hydroformylate many olefins. A raw material which is of importance in terms of quantity is propene, from which n- and i-butyraldehyde are obtained.

Industrial olefin mixtures which are used as feedstocks for the oxo process often comprise olefins having a variety of structures with different degrees of branching, different positions of the double bond in the molecule and possibly also different numbers of carbon atoms. A typical example is raffinate I, which is a mixture of the C₄-olefins 1-butene, 2-butene and isobutene. This is particularly true of olefin mixtures which have been formed by dimerization, trimerization or further oligomerization of C₂-C₅-olefins or other readily available higher olefins or by co-oligomerization of olefins. Examples of industrial olefin mixtures which can be hydroformylated to give the corresponding aldehyde mixtures are tripropene and tetrapropene and also dibutene, tributene and tetrabutene.

The products of the hydroformylation are determined by the structure of the starting olefins, the catalysts system and the reaction conditions. Under conditions under which no shift of the double bond in the olefin occurs, hereinafter referred to as nonisomerizating conditions, the formyl group is introduced at the place in the molecule where the double bond was located, which can result in two different products. Thus, for example, the hydroformylation of 1-pentene can form hexanal and 2-methylpentanal. In the hydroformylation under isomerizing conditions, under which a shift of the double bond in the olefin takes place in addition to the actual hydroformylation, 2-ethylbutanal would be expected as an additional product in the hydroformylation of 1-pentene.

If alcohols for the preparation of detergents and plasticizers are sought as downstream products of the oxo aldehydes, predominantly linear aldehydes should be prepared in the oxo process. The linear alcohols obtainable therefrom can be reacted to form the corresponding phthalates; these phthalates have particularly advantageous properties, e.g. a low viscosity. The abovementioned industrial olefin mixtures often contain only small proportions of olefins having a terminal double bond. To convert them into products in which more terminally hydroformylated olefin is present than there are olefins with a terminal double bond in the original olefin mixture, the hydroformylation has to be carried out under isomerizing conditions. Processes suitable for this purpose are, for example, high-pressure hydroformylations using cobalt catalysts. However, these processes have the disadvantage that they form relatively large amounts of by-products, for example alkanes, acetals or ethers.

When using rhodium complexes as catalyst for oxo reactions, the ligand also has a critical effect on the product composition of the aldehydes. Rhodium carbonyls without phosphorus-, arsenic- or nitrogen-containing ligands (unmodified rhodium catalysts) catalyze the hydroformylation of olefins having terminal and internal double bonds, which olefins may also be branched, to give aldehydes having a high degree of branching. The proportion of terminally hydroformylated olefin is significantly smaller than in the case of the cobalt-hydroformylated product.

In the presence of ligand-modified rhodium catalysts comprising rhodium and triorganophosphine, e.g. triphenylphosphine, a-olefins are terminally hydroformylated with high selectivity. Isomerization of the double bonds and/or hydroformylation of the internal double bonds hardly occurs at all. Using catalyst systems comprising bulky phosphite ligands, although isomerizing hydroformylation is achieved, the yields of terminally hydroformylated olefins which contain internal double bonds at branching sites are not satisfactory. An overview of the influence of ligands on the activity and selectivity in hydroformylation may be found in the above-cited reference by B. Cornils and W. A. Herrmann.

Compared to phosphine or phosphite ligands, the technical literature contains only few publications on the use of phosphorous diesters (hereinafter referred to as phosphonites) as ligands in hydroformylation reactions. WO 98/43935 describes catalyst systems comprising rhodium, a triorganophosphonite ligand or a bidentate phosphonite ligand for the hydroformylation of acyclic, cyclic olefins or olefin mixtures.

JP-A Hei 9-268152 discloses the used of acyclic phosphonite ligands for hydroformylation reactions. These acyclic ligands may only be prepared in a complex manner and are therefore unsuitable for an industrial process.

JP-A 9-255610 similarly describes the use of cyclic phosphonites. Here, a bisaryl system containing one phosphorus atom and one oxygen atom each forms a framework similar to phenanthrene to which an unsubstituted or substituted aryl radical is bound via a further oxygen atom. Systems of this type are still capable of improvement, based on the selectivity of hydroformylation reactions.

It is, therefore, an object of the present invention to provide a process for the hydroformylation of olefins using phosphonite ligands which enables branched, unbranched, terminal or internal olefins to be terminally hydroformylated in high yields and with high selectivities, i.e. it enables predominantly linear aldehydes to be prepared.

It has been found, surprisingly, that hydroformylations of olefins in the presence of catalysts of metal complexes, comprising a metal of transition group 8 and phosphonites, arsonites and stibonites leads to linear, terminally hydroformylated olefins in high yields and with high selectivities.

Thus, the present invention provides a process for the catalytic hydroformylation of olefins having from 3 to 24 carbon atoms, wherein the catalyst used comprises a metal of transition group 8 of the Periodic Table, in the presence of a ligand represented by formula I:

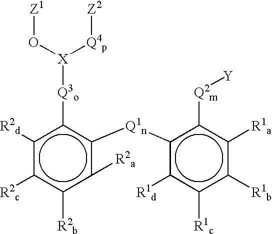

where

X=As, Sb, P;

R¹_{a-d}, R²_{a-d}=H, aliphatic or aromatic hydrocarbon radical, aliphatic or aromatic alkoxy group, in each case having from 1 to 25 carbon atoms, where R¹_{a-d} and R²_{a-d} can each be identical or different,

Q¹, Q², Q³, Q⁴=O, S, NR⁷, or CR⁷R⁸, where R⁷ and R⁸ can be identical or different and can have one of the meanings of R¹ₐ, with the proviso that either Q³ or Q⁴ is O, S, NR⁷;

n, m, o, p=0 or 1, with the proviso that either o or p is 1,

Y=—O—R⁵, —COOR⁵, —COOM, —SR⁵, —NR⁵R⁶, —N═CR⁵R⁶, —COR⁵, —CONR⁵R⁶, —F, —Cl, —Br, or —I, where R⁵ and R⁶ can be identical or different and are H, an aliphatic or aromatic hydrocarbon radical having from 1 to 25 carbon atoms, and M=H, Li, Na, K or NH₄

Z¹, Z²=substituted or unsubstituted aliphatic or aromatic hydrocarbon radical having from 1 to 75 carbon atoms, where Z¹ and Z² may be covalently linked.

### DETAILED DESCRIPTION OF THE INVENTION

A more complete appreciation of the invention and many of the attendant advantages thereof will be readily obtained as the same becomes better understood by reference to the following detailed description.

In particular embodiments of the present invention, ligands represented by formula II, III or IV may also be used:

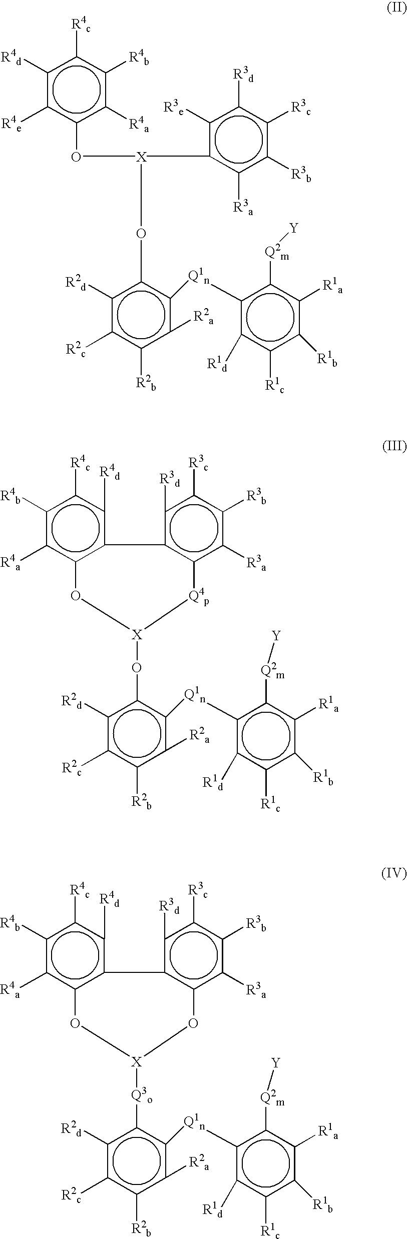

The radicals R¹_{a-d}, R²_{a-d}, R³ₐ₋ₑ and R⁴_{a-c} in these formulae are each H, aliphatic or aromatic hydrocarbon radical, an aliphatic or aromatic alkoxy group, in each case having from 1 to 25 carbon atoms, where R¹_{a-d}, R²_{a-d}, R³ₐ₋ₑ, R⁴ₐ₋ₑ can each be identical or different. Thus, for example, R¹ₐ can be a methyl group and R¹_{b} can be a methoxy group; this applies similarly to the radicals R²_{a-d}, R³ₐ₋ₑ, R⁴ₐ₋ₑ.

Q¹ and Q² are each O, S, NR⁷, a methylene radical CR⁷R⁸, where R⁷ and R⁸ can be identical or different and can have one of the meanings of R¹ₐ, Q³ and Q⁴ are each a methylene radical CR⁷R⁸, where R⁷ and R⁸ can be identical or different and can have a meaning of R¹ₐ. The indices n, m, o and p are each 0 or 1, if appropriate.

Y is —O—R⁵, —COOR⁵, —COOM, —SR⁵, —NR⁵R⁵, —N═CR⁵R⁶, —COR⁵, —CONR⁵R⁶, —F, —Cl, —Br, or —I, where R¹ and R⁶ can be identical or different and are H, an aliphatic or aromatic hydrocarbon radical having from 1 to 25 carbon atoms, where M=H, Li, Na, K or NH₄.

Ligands which can be used in the process of the invention include, for example, those shown in Table 1 below.

| | |
|---|---|
| | I-a |
| 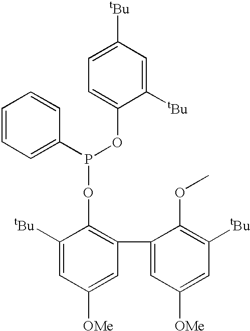 | |
| | I-b |
| 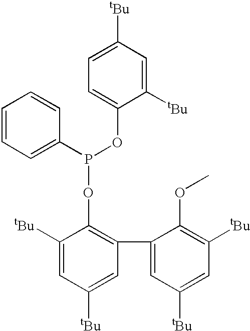 | |
| | I-c |
| 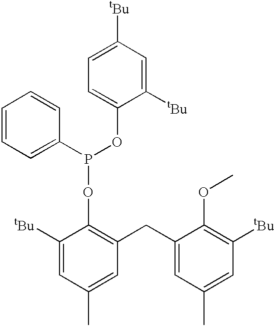 | |
| | I-d |
| 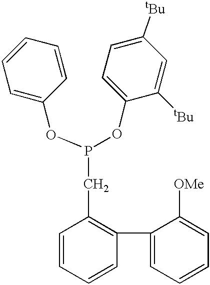 | |
| | I-e |
| 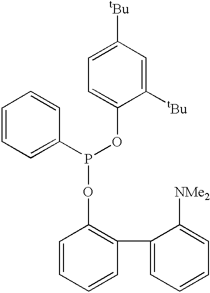 | |
| | I-f |
| 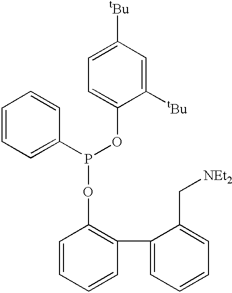 | |
| | I-g |
| 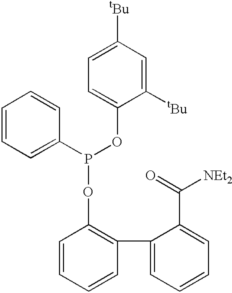 | |
| | I-h |
| 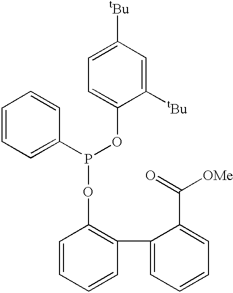 | |

| | |
|---|---|
| | II-a |
| 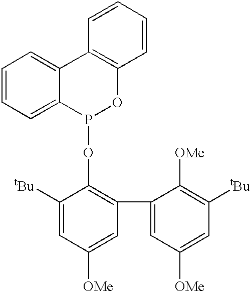 | |
| | II-b |
| 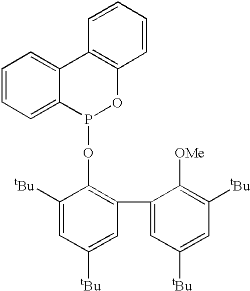 | |
| | II-c |
| 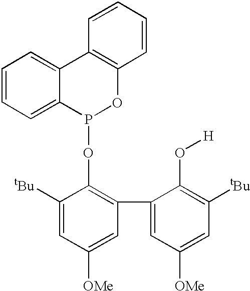 | |
| | II-d |
| 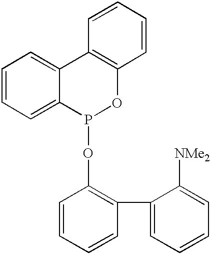 | |
| | II-e |
| 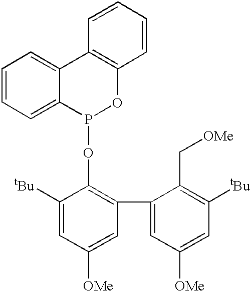 | |
| | II-f |
| 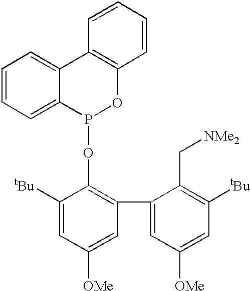 | |
| | II-g |
| 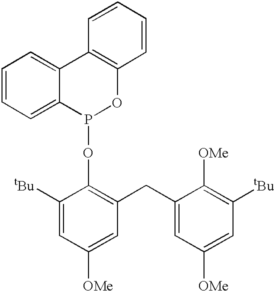 | |
| | II-h |
| 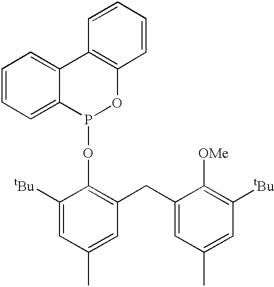 | |
| | II-i |
| 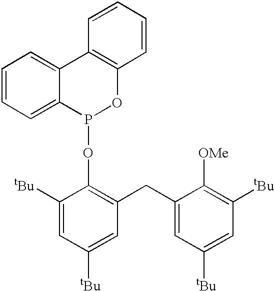 | |
| | II-j |
| 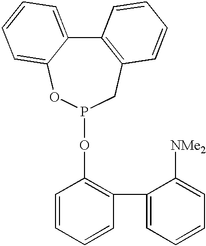 | |
| | II-k |
| 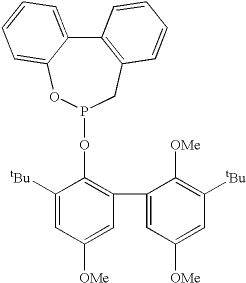 | |
| | II-l |
| 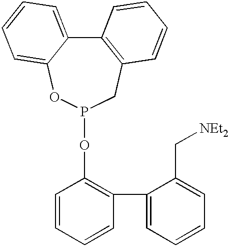 | |
| | II-m |
| 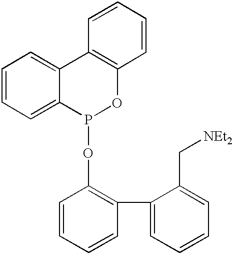 | |
| | II-n |
| 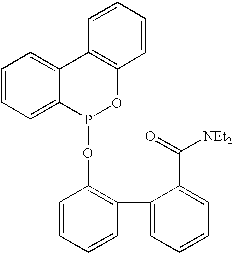 | |
| | III-a |
| 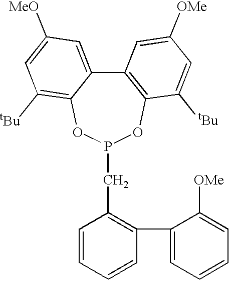 | |
| | III-b |
| 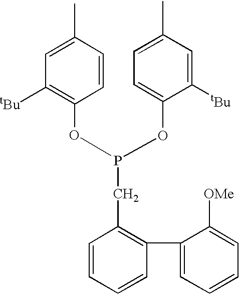 | |

The ligands of the formula I, II, III or IV used in the process of the invention will hereinafter be referred to as heterofunctionalized phosphonites, arsonites or stibonites. Ligands of this type may form hemilabile complexes with metal atoms of transition group 8 of the Periodic Table.

For the purposes of the present invention, these heterofunctionalized phosphonites, arsonites or stibonites are compounds containing an atom of main group V of the Periodic Table (P, As, Sb) which has one free electron pair and two single bonds each to an oxygen atom and one single bond to a carbon atom. The formulae I to IV and the examples in Table 1 show examples of ligands which may be used in the inventive process.

In addition to the atom of main group 5, the ligands contain at least one further heteroatom having at least one free electron pair. The atom of main group 5 and the further heteroatom are positioned in the ligand in such a way that a metal atom can be coordinated intramolecularly to both these atoms at the same time. This is the case when, for example, a phosphorus atom, a heteroatom and the intervening atoms can form a 4-15-, preferably an 8-12-membered ring, together with the coordinated metal atom. In the formulae I to IV, this ring can be formed by way of the metal of transition group 8, the atom X and the substituent Q²-Y.

The heteroatoms contained in the radical can be oxygen, sulfur, nitrogen, fluorine, chlorine, bromine or iodine. The heteroatoms may be present in functional groups such as ethers, thioethers and tertiary amines and/or be part of a chain or a ring. It is also possible for the ligands to contain more than one heteroatom which meets these requirements. The ligands used according to the invention should have a coordinate bond between heteroatom and metal which is less strong than that between the atom of main group V, i.e., P, As, Sb, and the metal.

In the technical literature, ligands which have a strong interaction with a metal together with a second, but distinctly weaker (labile) interaction are often referred to as hemilabile ligands (review articles: A. Bader, E. Linder, Coord. Chem. Rev. 1991, 108, 27-110; C. S. Slone, D. A. Weinberger, C. A. Mirkin, Prof. Inorg. Chem. 1999, 48, 233). In the case of some literature examples, the second, weaker interaction of the ligand with the metal has been able to be confirmed by means of X-ray structure analysis. In the case of the present heterofunctionalized ligands, the coordination behavior is not known but it can be concluded from steric considerations that it is possible for the metal to be coordinated both to, for example, an additional phosphorus atom and to an additional heteroatom.

The ligands of the formula I, II, III or IV used in the process of the invention are presumed to form a hemilabile bond by way of the group with the designation Y. The bisaryl substituent having the functional group Y represents an important feature of the ligands used in the process of the invention, since with these ligands hemilabile bonds can be formed to the central metal of the catalyst complex.

The process of the invention can be carried out with various catalysts and/or ligands.

Suitable catalytically active metals are the metals of transition group 8 of the Periodic Table of the Elements, for example rhodium, cobalt, platinum or ruthenium.

Here, the active catalyst complex for the hydroformylation is formed from a salt or a compound of the metal (catalyst precursor), the ligand and synthesis gas, which advantageously occurs in situ during the hydroformylation. Customary catalyst precursors are, for example, octanoates or acetylacetonates. The molar ratio of metal to ligand is from 1/1 to 1/1000, preferably from 1/1 to 1/50. The concentration of the metal in the reaction mixture is in the range from 1 ppm to 1000 ppm, preferably in the range from 5 ppm to 300 ppm. The reaction temperatures in the process of the invention are in the range from 60° C. to 180° C., preferably from 90° C. to 150° C., and the pressures are 1-300 bar, preferably 15-60 bar.

The catalyst, i.e. metal and ligand is homogeneously dissolved in the hydroformylation mixture comprising starting material (olefin) and the product (aldehydes, alcohols, high boilers). If desired, it is possible to use an additional solvent, for example, toluene, Texanol, high-boiling residues from the oxo process or phthalates such as di(2-ethylhexyl)phthalate.

The starting materials for a hydroformylation using the process of the invention are olefins or mixtures of olefins, in particular monoolefins having from 3 to 24, preferably from 4 to 16, particularly preferably from 3 to 12, carbon atoms and terminal or internal C—C double bonds, e.g. 1- or 2-pentene, 2-methyl-1-butane, 2-methyl-2-butene, 3-methyl-1-butane, 1-, 2- or 3-hexene, the C₆-olefin mixture obtained in the dimerization of propene (dipropene), heptenes, 2- or 3-methyl-1-hexene, octenes, 2-methylheptenes, 3-methylheptenes, 5-methyl-2-heptene, 6-methyl-2-heptene, 2-ethyl-1-hexene, the isomeric CH-olefin mixture obtained in the dimerization of butenes (dibutene), nonenes, 2- or 3-methyloctenes, the C₈-olefin mixture obtained in the trimerization of propene (tripropene), decenes, 2-ethyl-1-octane, dodecenes, the C₁₂-olefin mixture obtained in the tetramerization of propene or the trimerization of butenes (tetrapropene or tributene), tetradecenes, hexadecenes, the C₁₆-olefin mixture obtained in the tetramerization of butenes (tetrabutene) and olefin mixtures prepared by co-oligomerization of olefins having different numbers of carbon atoms (preferably from 2 to 4), if desired after fractional distillation to give fractions having the same or similar chain length. It is likewise possible to use olefins or olefin mixtures produced by the Fischer-Tropsch synthesis and also olefins which have been obtained by oligomerization of ethene or olefins which are obtainable via metathesis reactions. Preferred starting materials are C₄-, C₈-, C₁₂-, C₁₂- or C₁₆-olefin mixtures.

The process of the invention using the heterofunctionalized ligands makes it possible to hydroformylate α-olefins, branched, internal and internally branched olefins in high space-time yields. A notable aspect is the high yield of terminally hydroformylated olefin, even if only a small proportion of olefins having a terminal double bond was present in the starting material.

### EXAMPLES

Having generally described this invention, a further understanding can be obtained by reference to certain specific examples which are provided herein for purposes of illustration only and are not intended to be limiting unless otherwise specified.

### Example 1-17

### Hydroformylation of Octenes

30 ml of pure dry toluene, 1.875 mg (0.00604 mmol) of [acacRh(COD)] (rhodium cyclooctadienylacetylacetonate), dissolved in 10 ml of toluene, and 0.00604 or 0.01208 mmol of the respective ligand dissolved in 1 ml of toleune were placed into a 200 ml autoclave under a protective gas. 15 ml (10.62 g, 94.63 mmol) of octene mixture (see Table 2 for composition) were placed into a pressure pipette over the reactor. Reactor and pressure pipette were charged to 33 bar of CO/H₂ (1/1 synthesis gas) via a bypass connected in parallel to the pressure-control section and the reactor contents were brought to the reaction temperature with stirring via a sparging stirrer at 1500 rpm. After the pressure had been increased to 45 to 47 bar, the olefin mixture was forced from the pressure pipette into the reactor. The intended temperature and pressure set-point were set. The bypass was closed and the pressure was kept constant (50 bar for the Examples 1-11) over the entire reaction time using a pressure controller. The experiment was terminated with forced cooling when the gas consumption rates observed using a gas flow meter fell below 2 ml/mint The reaction solution was taken off under protective gas and analyzed by gas chromatography.

For the Examples 1-11 summarized in Table 3, two mixtures (A and B) of octenes were used (see Table 2 for composition). The numbering of the phosphonite ligands used (Ia, Ib, Ia, IIb, IIc) corresponds to that in Table 1.

TABLE 2

| | A (% by weight) | B (% by weight) |
|---|---|---|
| n-1-Octene | 9.8 | 3.4 |
| cis + trans-2-Octene | 70.0 | 49.8 |
| cis + trans-3-Octene | 15.5 | 30.0 |
| cis + trans-4-Octene | 4.7 | 16.8 |

TABLE 3

| Example | 1 | 2 | 3 | 4 | 5 | 6 | 7 | 8 | 9 | 10 |
|---|---|---|---|---|---|---|---|---|---|---|
| Ligand | I-a | I-a | I-a | I-a | I-a | II-a | II-a | II-a | II-a | II-a |
| Octene mixture | A | A | A | A | A | A | A | A | A | A |
| P/Rh | 1/1 | 2/1 | 1/1 | 1/1 | 1/1 | 1/1 | 2/1 | 1/1 | 1/1 | 1/1 |
| Temperature | 120 | 120 | 140 | 120 | 120 | 120 | 120 | 140 | 120 | 140 |
| (° C.) | | | | | | | | | | |
| Pressure (bar) | 50 | 50 | 50 | 50 | 50 | 50 | 50 | 50 | 50 | 50 |
| Time (h) | 4 | 4 | 4 | 4 | 3 | 3 | 2 | 3 | 3 | 3 |
| Composition of the aldehydes in % | | | | | | | | | | |
| Nonanal | 29.5 | 29.2 | 31.8 | 28.5 | 29.7 | 29.1 | 27.4 | 33.0 | 30.6 | 32.0 |
| 2-Methyloctanal | 39.3 | 39.4 | 37.9 | 38.8 | 39.4 | 38.4 | 38.1 | 37.1 | 38.6 | 37.6 |
| 3-Ethylheptanal | 16.6 | 16.7 | 16.1 | 17.2 | 16.5 | 17.5 | 18.6 | 15.9 | 16.6 | 15.6 |
| 4-Propylhexanal | 14.6 | 14.7 | 14.2 | 15.5 | 14.4 | 15.0 | 15.9 | 14.0 | 14.2 | 14.8 |
| Yield in % | 89 | 94 | 91 | 95 | 98 | 98 | 97 | 99 | 99 | 88 |

| Example | 11 | 12 | 13 | 14 | 15 | 16 | 17 | Comparison |
|---|---|---|---|---|---|---|---|---|
| Ligand | II-a | II-c | II-c | II-c | II-c | II-c | II-c | phosphite |
| Octene mixture | B | B | B | B | B | B | B | B |
| P/Rh | 1/1 | 1/1 | 5/1 | 10/1 | 20/1 | 50/1 | 50/1 | 20/1 |
| Temperature | 120 | 140 | 140 | 140 | 140 | 140 | 140 | 140 |
| (° C.) | | | | | | | | |
| Pressure (bar) | 50 | 20 | 20 | 20 | 20 | 20 | 20 | 20 |
| Time (h) | 3 | 6 | 6 | 6 | 6 | 6 | 6 | 6 |
| Composition of aldehydes in % | | | | | | | | |
| Nonanal | 29.1 | 31.7 | 41.0 | 47.8 | 45.9 | 47.9 | 41.8 | 24.5 |
| 2-Methyloctanal | 37.3 | 38.4 | 33.7 | 30.0 | 30.4 | 27.6 | 31.9 | 37.4 |
| 3-Ethylheptanal | 17.7 | 15.9 | 13.5 | 11.8 | 12.6 | 12.9 | 14.0 | 21.2 |
| 4-Propylhexanal | 15.9 | 14.0 | 11.8 | 10.4 | 11.1 | 11.6 | 12.3 | 16.9 |
| Yield in % | 98 | 21.0 | 37.7 | 51.9 | 76.6 | 86.0 | 72.7 | 89.3 |

Note on Example 17:

Three times the olefin concentration, inverse experimental procedure: olefin introduced and heated, Rh and ligand dissolved in toluene, added from pipette.

### Comparison Example

Hydroformylation was carried out under the conditions of Example 12, but instead of the heterofunctionalized phosphonite, a phosphite ligand (tris[2,4-ditertbutylphenyl]phosphite) was used. The proportion of nonanal in the total amount of aldehyde was 24.5%.

### Examples 18-21

### Hydroformylation of di-n-butene

Experiments 18-21 were carried out in a similar manner to Experiments 1-17. The olefin used was dimerized n-butene (di-n-butene). The content of olefin having a terminal double bond (essentially 1-octene, 3-methyl-1-heptene, 5-methyl-1-heptene, 2-ethyl-1-hexene, 3,4-dimethyl-1-hexene, 2-ethyl-3-methyl-1-pentene) was less than 5%.

The experiments were terminated in each case after 8 h.

TABLE 4

| Example | 18 | 19 | 20 | 21 |
|---|---|---|---|---|
| Temperature | 140 | 140 | 150 | 130 |
| (° C.) | | | | |
| Pressure (bar) | 20 | 20 | 30 | 30 |
| Time (h) | 8 | 8 | 8 | 8 |
| Rh concen- | 20 | 150 | 150 | 20 |
| tration | | | | |
| (ppm) | | | | |
| P/Rh | 10 | 10 | 10 | 10 |
| Olefin | di-n-butene | di-n-butene | di-n-butene | di-n-butene |
| Ligand | II-a | II-a | II-a | II-a |
| Conversion | 19.5 | 30.2 | 52.4 | 29.0 |
| rate (%) | | | | |
| n %* | 41.3 | 40.3 | 38.8 | 35.4 |

*Proportion of the aldehyde produced by terminal hydroformation (essentially nonanal, 4-methyloctanal, 3-ethylheptanal, 6-methyloctanal, 4,5-dimethylheptanal) of the total amount of aldehydes formed.

In Examples 18-21, it is apparent that using the novel catalyst systems, even in the case of hydroformylation of technical-grade olefin mixtures which principally comprise branched olefins having internal double bonds, a high proportion of terminally hydroformylated product is obtained.

Obviously, numerous modifications and variations of the present invention are possible in light of the above teachings. It is therefore to be understood that within the scope of the appended claims, the invention may be practiced otherwise than as specifically described herein.

This application is based on German Patent Application Serial No. 19954721.1, filed on Nov. 12, 1999, and incorporated herein by reference in its entirety.

1. A process for the catalytic hydroformylation of olefins having from 3 to 24 carbon atoms, comprising:
hydroformylating at least one olefin having from 3 to 24 carbon atoms in the presence of a catalyst wherein the catalyst comprises a metal of transition group 8 of the Periodic Table and a ligand represented by formula I: 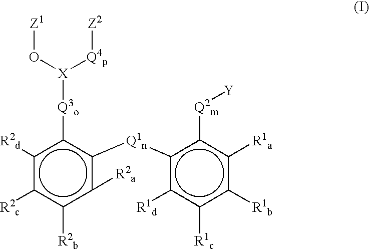
wherein
X=As, Sb, or P;
R¹_{a-d}, R²_{a-d}=H, an aliphatic or aromatic hydrocarbon radical, or an aliphatic or aromatic alkoxy group, in each case having from 1 to 25 carbon atoms, where R¹_{a-d} and R⁴_{a-d} are identical or different;
Q¹, Q², Q³, Q⁴=O, S, NR⁷, or CR⁷R⁸, where R⁷ and R⁸ can be identical or different and can have one of the meaning of R¹ₐ, with the proviso that either Q³ or Q⁴ is O, S, or NR⁷;
n, m, o, p=0 or 1, with the proviso that either o or p is 1;
Y=—O—R⁵, —COOR⁵, —COOM, —SR⁵, —NR⁵R⁶, —N═CR⁵R⁶, —COR⁵, —CONR⁵R⁶, —F, —Cl —Br, or —I,
where R⁵ and R⁶ are identical or different and are H, or an aliphatic or aromatic hydrocarbon radical having from 1 to 25 carbon atoms, and M is H, Li, Na, K or NH₄,
Z¹, Z²=a substituted or unsubstituted aliphatic or aromatic hydrocarbon radical having from 1 to 75 carbon atoms, where Z¹ and Z² can be covalently linked.

2. The process of claim 1, wherein the ligand is represented by formula II: 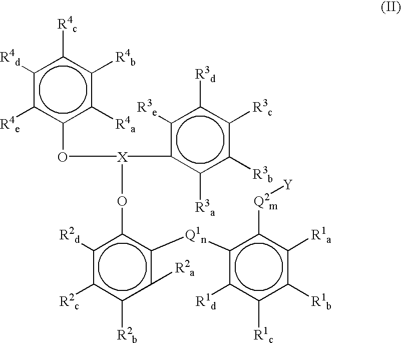
wherein X=As, Sb, or P;
R¹_{a-d}, R²_{a-d}, R³ₐ₋ₑ, R⁴ₐ₋ₑ=H, an aliphatic or aromatic hydrocarbon radical, or an aliphatic or aromatic alkoxy group, in each case having from 1 to 25 carbon atoms, where R¹_{a-d}, R⁴_{a-d}, R³ₐ₋ₑ and R⁴ₐ₋ₑ are identical or different;
Q¹, Q²=O, S, NR⁷, or CR⁷R⁸, where R⁷ and R⁸ are identical or different and are as defined for R¹ₐ;
n, m=0 or 1; and
Y=—O—R⁵, —COOR⁵, —COOM, —SR⁵, —NR⁵R⁶, —N═CR⁵R⁵, —COR⁵, —CONR⁵R⁶, —F, —Cl, —Br, or —I, where R⁵ and R⁶ can be identical or different and are H, an aliphatic or aromatic hydrocarbon radical having from 1 to 25 carbon atoms, where M=H, Li, Na, K or NH₄.

3. The process of claim 1, wherein the ligand is represented by formula III: 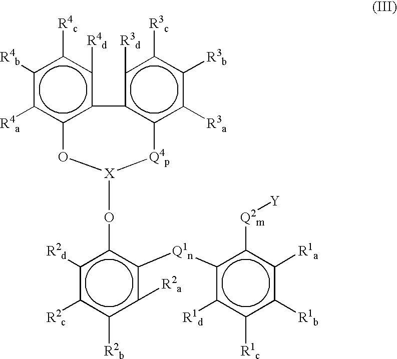
wherein
X=As, Sb, or P;
R¹_{a-d}, R²_{a-d}, R³_{a-d}, R⁴_{a-d}=H, an aliphatic or aromatic hydrocarbon radical, an aliphatic or aromatic alkoxy group, in each case having from 1 to 25 carbon atoms, where R¹_{a-d}, R²_{a-d}, R³_{a-d}, R⁴_{a-d} are identical or different;
Q¹, Q²=O, S, NR⁷, or CR⁷R⁸, where R⁷ and R⁸ are identical or different and are as defined for R¹ₐ;
Q⁴=CR⁷R⁸, where R⁷ and R⁸ are identical or different and are as defined as for R1ₐ,
n, m, p=0 or 1; and
Y=—O—R⁵, —COOR⁵, —COOM, —SR⁵, —NR⁵R⁶, —N═CR⁵R⁶, —COR⁵, —CONR⁵R⁶, —F, —Cl, —Br, or —I, where R⁵ and R⁶ are identical or different and are H, or an aliphatic or aromatic hydro-carbon radical having from 1 to 25 carbon atoms, where M=H, Li, Na, K or NH₄.

4. The process of claim 1, wherein the ligand is represented by formula IV: 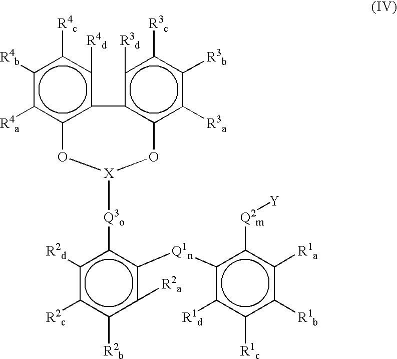
wherein
X=As, Sb, or P;
R¹_{a-d}, R²_{a-d}, R³_{a-d}, R⁴_{a-d}=H, an aliphatic or aromatic hydro-carbon radical, or an aliphatic or aromatic alkoxy group, in each case having from 1 to 25 carbon atoms, where R¹_{a-d}, R²_{a-d}, R³_{a-d}, R⁴_{a-d} are identical or different;
Q¹, Q²=O, S, NR⁷, or CR⁷R⁸, where R⁷ and R⁸ are identical or different and are as defined for R¹ₐ;
Q³=CR⁷R⁸, where R⁷ and R⁸ are identical or different and are as defined for R¹ₐ;
n, m, o=0 or 1; and
Y=—O—R⁵, —COOR⁵, —COOM, —SR⁵, —NR⁵R⁶, —N═CR⁵R⁶, —COR⁵, —CONR⁵R⁶, —F—Cl—Br, or —I, where R⁵ and R⁶ are identical or different and are H, or an aliphatic or aromatic hydro-carbon radical having from 1 to 25 carbon atoms, where M=H, Li, Na, K or NH₄.

5. The process of claim 1, wherein the metal of transition group 8 of the Periodic table is cobalt or rhodium.

6. The process of claim 1, where in the olefin has from 3 to 8 carbon atoms.

7. The process of claim 1, wherein a mixture of olefins is hydroformylated.

8. The process of claim 1, wherein the hydroformylation is conducted at a temperature of 60° C. to 180° C.

9. The process of claim 1, wherein the hydroformylation is conducted at a pressure of 1-300 bar.
